(19) United States
(12) Patent Application Publication
(10) Pub. No.: US 2012/0157792 A1
(43) Pub. Date: Jun. 21, 2012

(54) CARDIOVASCULAR HEALTH STATUS EVALUATION SYSTEM AND METHOD

(76) Inventors: Chia-Chi CHANG, Hsinchu (TW); Tsu-Chien Hsiao, Hsinchu (TW); Hung-Yi Hsu, Taichung City (TW)

(21) Appl. No.: 12/971,796

(22) Filed: Dec. 17, 2010

(51) Int. Cl. A61B 5/00

(52) U.S. Cl. 600/301

(57) ABSTRACT

A cardiovascular health status evaluation system includes a sensing unit and a computing unit. The sensing unit is for measuring impulses of the cardiovascular circulation of a user. The computing unit is for calculating a standard deviation and an average of time intervals between the impulses in a unit time, and based on a change of a ratio between the standard deviation and average, evaluating the cardiovascular health status of the user. The evaluation index of the aforementioned system includes a personalized parameter (the average), thereby enabling a personalized comparison. A cardiovascular health status evaluation method is also disclosed herein.

### BACKGROUND OF THE INVENTION

1. Field of the Invention

The present invention relates to a cardiovascular health status evaluation system and method, and more particularly to a cardiovascular health status evaluation system and method for living care.

2. Description of the Prior Art

Medical institutes typically adopt cardiovascular health status evaluation methods that involve measuring for cardiovascular circulation data with a precision instrument, and converting the measured data into an evaluation index to evaluate the cardiovascular health status of a user. For example, baro-reflex sensitivity (BRS) index, commonly used by medical institutes at present, requires measuring a user's blood pressure continuously. The requirement of an expensive equipment hinders the popularity of the cardiovascular health status evaluation system adopted by medical institutes among normal families.

Currently, a typical living care medical instrument first measures for physiological data of a user, and then converts the physiological data into an evaluation index to be compared with a normal reference value listed on a user's manual for self-evaluation of health status. The normal reference value is a statistical value obtained from ample tests and observations, and is a collective result. However, the prior art living care medical instrument cannot provide personalized comparison that accounts for individual differences between users. In addition, the prior art living care medical instrument does not provide an evaluation index that corresponds to what is being used by medical institutes. In other words, the medical institutes cannot obtain the required data from the provided data by the user for an initial assessment. Therefore, the medical institutes would have to re-measure for the physiological data of the user, causing a longer diagnostic and treatment time.

In summary, it is highly desirable to provide a cardiovascular health status evaluation system and method for living care that feature a personalized comparison.

### SUMMARY OF THE INVENTION

The present invention provides a cardiovascular health status evaluation system and method that measures the impulses of cardiovascular circulation of a user, calculates a standard deviation and an average of time intervals between the impulses, and based on a change of a ratio between the standard deviation and average, evaluates the cardiovascular health status of the user. Since the evaluation index includes personalized physiological data, i.e. the average, the system and method of the present invention feature a personalized comparison, and are suitable for cardiovascular health status evaluation for living care.

According to an embodiment, the cardiovascular health status evaluation system includes a sensing unit and a computing unit. The sensing unit is for measuring impulses of cardiovascular circulation of a user. The computing unit electrically connected with the sensing unit is for calculating a standard deviation and an average of the impulses of the cardiovascular circulation in a unit time, and based on a change of a ratio between the standard deviation and the average, evaluating the cardiovascular health status of the user.

In accordance with another embodiment, the cardiovascular health status evaluation method includes: measuring impulses of cardiovascular circulation of a user; calculating a standard deviation and an average of time intervals between impulses of the cardiovascular circulation in a unit time; and evaluating the cardiovascular health status of the user based on a change of a ratio between the standard deviation and the average.

The objective, technologies, features and advantages of the present invention will become more apparent from the following description in conjunction with the accompanying drawings, wherein certain embodiments of the present invention are set forth by way of illustration and examples.

### BRIEF DESCRIPTION OF THE DRAWINGS

FIG. 1 is a block diagram illustrating the cardiovascular health status evaluation system according to an embodiment of the present invention;

FIG. 2a is a correlation diagram illustrating the correlations between the standard deviation and BRS index respectively for cases where healthy subjects are in a lying-down position and a tilting position;

FIG. 2b is a correlation diagram illustrating the correlations between the ratio of the standard deviation and average, and BRS index respectively for cases where healthy subjects are in a lying-down position and a tilting position;

FIG. 3a is a correlation diagram illustrating the correlations between the standard deviation and BRS index respectively for healthy subjects and stroke subjects in a lying-down position;

FIG. 3b is a correlation diagram illustrating the correlations between the ratio of the standard deviation and average, and BRS index respectively for healthy subjects and stroke subjects in a lying-down position; and

FIG. 4 is a flow chart illustrating the cardiovascular health status evaluation method according to an embodiment of the present invention.

### DESCRIPTION OF THE PREFERRED EMBODIMENT

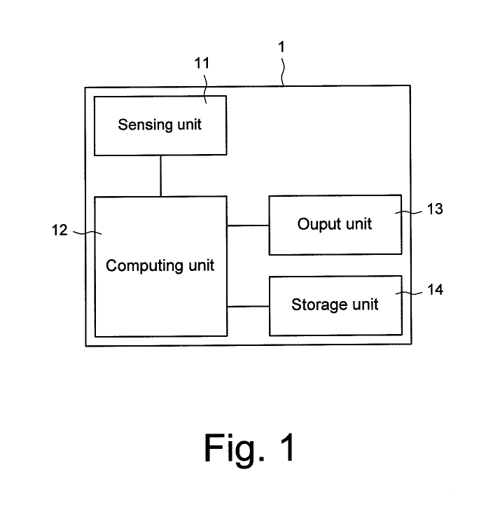

Referring to FIG. 1, the cardiovascular health status evaluation system 1 according to an embodiment includes a sensing unit 11 and a computing unit 12. The sensing unit 11 is for measuring impulses of the cardiovascular circulation of a user. For example, the sensing unit 11 may measure the heart beat, pulse, blood oxygen concentration or the combination thereof to obtain the impulses of the cardiovascular circulation of the user. According to an embodiment, the sensing unit 11 may be an electrocardiograph (ECG), a pulse oximeter (SpO₂), a photo-plethysmograph (PPG) or the combination thereof.

Continuing the above description, the computing unit 12 is electrically connected with the sensing unit 11 for calculating a standard deviation and an average of time intervals between the impulses of the cardiovascular circulation in a unit time. For instance, the sensing unit 11 may continuously measure the impulses of the cardiovascular circulation of the user for the unit time such as 5 minutes. The computing unit 12 calculates the average and standard deviation of the time intervals between the impulses within the 5 minutes, and then evaluates the cardiovascular health status based on a change of a ratio between the standard deviation and average. According to an embodiment, the sensing unit 11 and the computing unit 12 may be realized as an SoC (system on chip) to lower the cost and reduce the size, thereby increasing its popularity to common families.

According to an embodiment, the cardiovascular health status evaluation system 1 may include an output unit 13 electrically connected with the computing unit 12. The output unit 13 may output the cardiovascular health status of the user evaluated by the computing unit 12. For example, the output unit 13 may be a display, an indicator light, a printer, a speaker or the combination thereof.

When the impulses of the cardiovascular circulation are measured, the body of the user is under homeostatic control and hence, the standard deviation of the time intervals between the impulses may reflect the quality of feedback control. Normally speaking, given that the user has no abnormality such as arrhythmia, a larger standard deviation for the time intervals between the impulses may be construed as having a better feedback control. Because the average of time intervals between impulses is different for each individual user, the change of the ratio between the standard deviation and average would be from a personalized comparison.

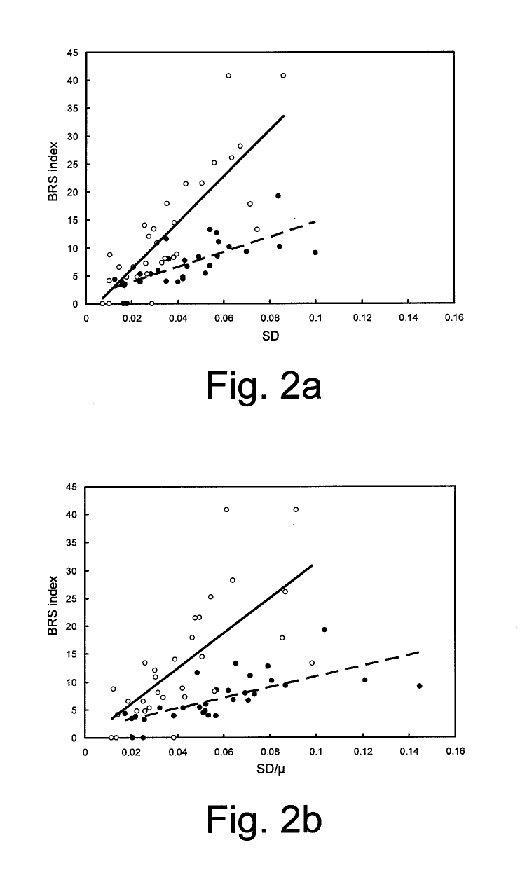

Referring to FIG. 2a and FIG. 2b, FIG. 2a is a correlation diagram for the standard deviation (SD) and the baro-reflex sensitivity (BRS) index used by medical institutes; FIG. 2b is a correlation diagram for the ratio of the standard deviation (SD) and average (μ), and the baro-reflex sensitivity (BRS) index. FIG. 2a and FIG. 2b illustrate the test results from healthy subjects in a lying-down position and a tilting position, wherein the hollow circles are test results for lying-down position, and the solid line is the regression line thereof; and the solid circles are test results for tilting position, and the dashed line is the regression line thereof. Since the ratio of the standard deviation and average and the BRS index are highly correlated, the computing unit 12 may correspond the ratio of the standard deviation and average to the BRS index used by medical institutes to evaluate the cardiovascular health status of the user. In other words, the medical institutes can also refer to the evaluation index of the present embodiment for an initial assessment.

Moreover, FIG. 2a and FIG. 2b illustrate the test results of healthy subjects under a lying-down position (first state) and a tilting position (second state), and the evaluation indices for the tilting position shift to the right both in FIG. 2a and FIG. 2b. Therefore, when the evaluation index shown in FIG. 2b, i.e. the ratio of the standard deviation and average, right shifts, it indicates that the feedback control of the evaluated user is effective, and the health status is relatively better.

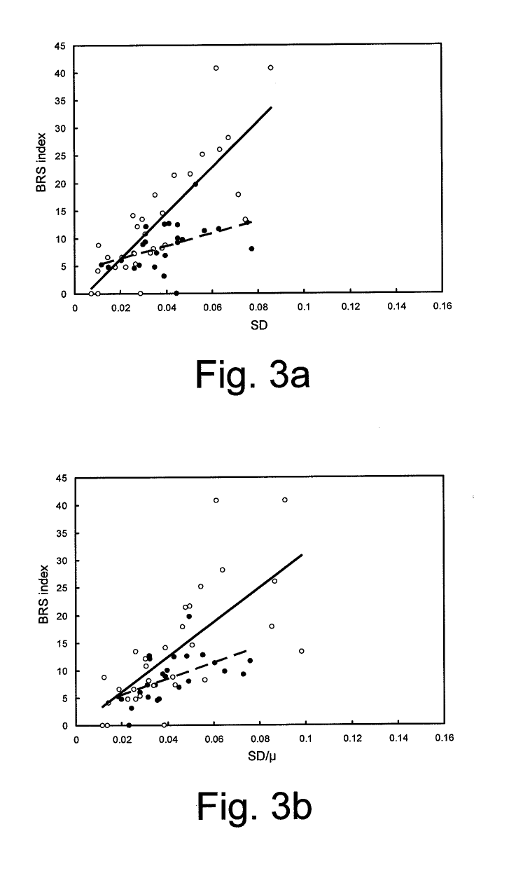

Referring to FIG. 3a and FIG. 3b, FIG. 3a is a correlation diagram for the standard deviation (SD) and the BRS index; FIG. 3b is a correlation diagram for the ratio of the standard deviation (SD) and average (p), and the BRS index. In FIG. 3a and FIG. 3b, hallow circles are test results of healthy subjects under the lying-down position, and the solid line is the regression line thereof; solid circles are test results of stroke subjects under the lying-down position, and the dashed line is the regression line thereof. As shown in FIG. 3b, compared to the evaluation indices of the healthy subjects, those of stroke subjects clearly shift to the left. Hence, when the evaluation index shown in FIG. 3b, i.e. the ratio of the standard deviation and average, left shifts, it indicates that the feedback control of the user is less effective, and the health status is relatively worse. In such case, the cardiovascular health status evaluation system 1 of the present embodiment may suggest the user to be further examined by a medical institute for further health evaluation.

In addition, referring to FIG. 2a and FIG. 3a, in the case where only the standard deviation is used as the evaluation index, the differences between healthy subjects and stroke subjects are less significant. Referring to FIG. 2b and FIG. 3b, when the ratio of the standard deviation and average is used as the evaluation index, more significant differences between healthy subjects and stroke subjects are observed, showing that the evaluation index of the present invention is suitable for a personalized comparison.

According to an embodiment, the cardiovascular health status evaluation system 1 may include a storage unit 14 electrically connected with the computing unit 12. The storage unit 14 may store the evaluation result from the computing unit 12, such as the ratio of the standard deviation and average. It is noted that separately storing the standard deviation and average is equivalent to storing the ratio of the standard deviation and average directly. Based on this configuration, the computing unit 12 may compare the ratio obtained presently with that stored in the storage unit 14 obtained previously, and evaluates the cardiovascular status of the user based on whether the ratio shifts to the left or right.

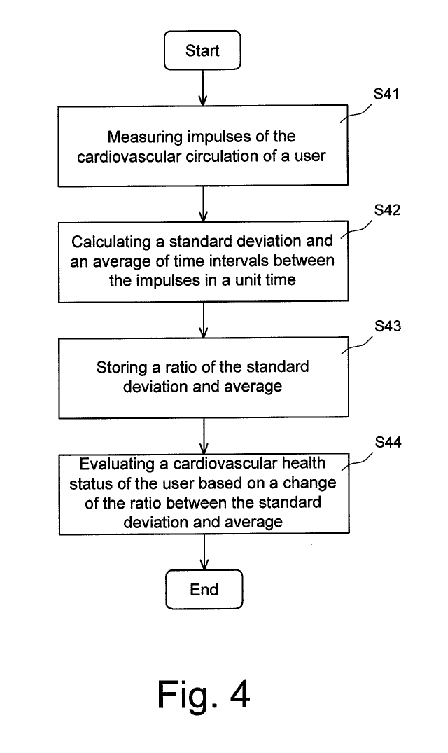

Referring to FIG. 4, the cardiovascular health status evaluation method according to an embodiment includes: measuring impulses of cardiovascular circulation of a user (S41); calculating a standard deviation and an average of time intervals between the impulses in a unit time (S42); and evaluating a cardiovascular health status of the user based on a change of a ratio between the standard deviation and average (S44). According to an embodiment, the cardiovascular health status evaluation method may store the ratio of the standard deviation and average obtained by step S42 for future reference (S43) to evaluate if the user's cardiovascular health status has changed. In accordance with an embodiment, the cardiovascular health status evaluation method includes outputting the cardiovascular health status evaluated by step S44 for user's reference. Other steps have been described in detail above and the description thereof are omitted here.

In conclusion, the cardiovascular health status evaluation system and method of the present invention may employ a low cost hardware to measure impulses of cardiovascular circulation of a user, calculate a standard deviation and an average of time intervals between the impulses, and based on a change of a ratio between the standard deviation and average, evaluate the cardiovascular health status of the user. Thus, the system and method of the present invention may feature personalized comparison and may be suitable for cardiovascular health status evaluation for living care. Besides, the evaluation index of the present invention may correspond to that of medical institutes, and may be used for initial assessment by medical institutes.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A cardiovascular health status evaluation system comprising:
a sensing unit for measuring impulses of cardiovascular circulation of a user; and
a computing unit electrically connected with the sensing unit for calculating a standard deviation and an average of the impulses of the cardiovascular circulation in a unit time, and based on a change of a ratio between the standard deviation and the average, evaluating the cardiovascular health status of the user.

2. The cardiovascular health evaluation system according to claim 1, wherein the computing unit corresponds the ratio to a baro-reflex sensitivity (BRS) index to evaluate the cardiovascular health status of the user.

3. The cardiovascular health evaluation system according to claim 1, further comprising:
a storage unit electrically connected with the computing unit for storing the ratio.

4. The cardiovascular health evaluation system according to claim 3, wherein the computing unit further compares for the change between the previous and present calculated ratios to evaluate the cardiovascular health status of the user.

5. The cardiovascular health evaluation system according to claim 1, wherein the computing unit compares for the change between the ratios respectively when the user is under a first state and a second state to evaluate the cardiovascular health status of the user.

6. The cardiovascular health evaluation system according to claim 1, wherein the sensing unit measures the heart beat, pulse, blood oxygen concentration or the combination thereof.

7. The cardiovascular health evaluation system according to claim 1, wherein the sensing unit comprises an electrocardiograph, a pulse oximeter, a photo-plethysmograph or the combination thereof.

8. The cardiovascular health evaluation system according to claim 1, further comprising:
an output unit electrically connected with the computing unit for outputting the cardiovascular health status of the user evaluated by the computing unit.

9. The cardiovascular health evaluation system according to claim 8, wherein the output unit comprises a display, an indicator light, a printer, a speaker or the combination thereof.

10. A cardiovascular health status evaluation method comprising:
measuring impulses of cardiovascular circulation of a user;
calculating a standard deviation and an average of time intervals between impulses of the cardiovascular circulation in a unit time; and
evaluating the cardiovascular health status of the user based on a change of a ratio between the standard deviation and the average.

11. The cardiovascular health status evaluation method according to claim 10, wherein the evaluating step corresponds the ratio to a baro-reflex sensitivity (BRS) index to evaluate the cardiovascular health status of the user.

12. The cardiovascular health status evaluation method according to claim 10, further comprising:
storing the ratio.

13. The cardiovascular health status evaluation method according to claim 12, further comprising:
comparing for the change between the previous and present calculated ratios to evaluate the cardiovascular health status of the user.

14. The cardiovascular health status evaluation method according to claim 10, wherein the evaluating step further compares for the change between the ratios respectively when the user is under a first state and a second state to evaluate the cardiovascular health status of the user.

15. The cardiovascular health status evaluation method according to claim 10, wherein the measuring step measures the heart beat, pulse, blood oxygen concentration or the combination thereof.

16. The cardiovascular health status evaluation method according to claim 10, wherein the impulses of the cardiovascular circulation are measured by an electrocardiograph, a pulse oximeter, a photo-plethysmograph or the combination thereof.

17. The cardiovascular health status evaluation method according to claim 10, further comprising:
outputting the evaluated cardiovascular health status.
